(19) United States
(12) Patent Application Publication
(10) Pub. No.: US 2022/0401383 A1
(43) Pub. Date: Dec. 22, 2022

(54) Cannabidiol and/or Cobicistat Combination Drug Therapy

(71) Applicant: SHINKEI THERAPEUTICS, INC., Princeton, NJ (US)

(72) Inventors: Suresh Borsadia, Plainsboro, NJ (US); Kalpana Patel, West Windsor, NJ (US); Krunal RAVAL, Ahmedabad (IN)

(21) Appl. No.: 17/779,122

(22) PCT Filed: Nov. 23, 2020
(86) PCT No.: PCT/US20/61848

§ 371 Date: May 23, 2022

Related U.S. Application Data
(60) Provisional application No. 62/938,877, filed on Nov. 21, 2019.

(51) Int. Cl. A61K 31/05; A61K 31/5377; A61K 45/06

(52) U.S. Cl. CPC A61K 31/05; A61K 31/5377; A61K 45/06

(57) ABSTRACT

Compositions comprising cannabidiol and one or more compounds which are inhibitors of CYP2C19 and CYP3A4 enzymes are disclosed. Methods of treating pain, epilepsy, sleep deprivation, vomiting, nausea, psychosis, anxiety, depression, movement disorders, and other neuropsychiatric or neurogenic diseases using such compositions are also provided. Compositions comprising cobicistat, or pharmaceutically acceptable isomers, salts, and/or solvates thereof; and at least one therapeutic agent which is metabolized by CYP2C19, and optionally CYP3A4, are provided. Methods for inhibiting CYP2C19, and optionally CYP3A4, in a patient in need thereof are also provided, comprising administering to the patient an amount of cobicistat, or pharmaceutically acceptable isomers, salts, and/or solvates thereof, effective for inhibiting CYP2C19, and optionally CYP3A4, in the patient.

### FIELD OF THE INVENTION

Compositions comprising cannabidiol and one or more compounds which are inhibitors of CYP2C19 and CYP3A4 enzymes are disclosed. Compositions comprising cobicistat and at least one therapeutic agent which is metabolized by CYP2C19 enzyme are disclosed. Methods of treatment using such compositions are also provided.

### BACKGROUND

CYP3A4 and CYP2C19 are members of the cytochrome P450 (CYP) family of oxidizing enzymes. The cytochrome P450 proteins are monooxygenases that catalyze many reactions involved in drug metabolism and synthesis of cholesterol, steroids, and other lipids components. Many drugs may increase or decrease the activity of various CYP isozymes either by inducing the biosynthesis of an isozyme (enzyme induction) or by directly inhibiting the activity of the CYP (enzyme inhibition). CYP2C19 is a liver enzyme that acts on at least 10% of commonly prescribed drugs and is involved in the metabolism of xenobiotics, including many proton pump inhibitors and antiepileptics. CYP3A4 is the most abundantly expressed human CYP enzyme, mainly found in the liver and intestine, and metabolizes about 30% to 50% of marketed drugs.

Plant derived cannabidiol (CBD), Epidiolex®, has recently been approved by United States Food and Drug Administration (FDA) for the treatment of seizures associated with Lennox—Gastaut syndrome (LGS) or Dravet syndrome (DS) in patients 2 years of age and older (Epidiolex® label, Greenwich Biosciences, Inc., 2008, pg. 1). CBD is the main non-psychoactive constituent of Cannabis sativa, accounting for up to 40% of the plant's extract. Campos et al., Philos Trans R Soc Lond B Biol Sci. 2012;367(1607):3364-78. CBD is currently being investigated for the treatment of a variety of anxiety, cognition, and movement disorders, as well as for the treatment of pain. CBD has shown promise for treatment of a wide range of conditions, such as for epilepsy, sleep deprivation, vomiting, nausea, psychosis, anxiety, depression, movement disorders, and other neuropsychiatric or neurogenic diseases; and for pain relief in patients suffering from headache, migraines, rheumatoid arthritis, neuropathy, allodynia, overactive bladder, spasticity, multiple sclerosis, HIV, glioblastoma, cancer, and other acute or chronic pain conditions. Urits et al., Pain Ther 2019; 8(1):41-51.

CBD has significant adverse drug reaction which promotes intolerance. Epidiolex® label (pg. 8) reports significant difference in adverse drug reactions between CBD and placebo treatment as shown in below table, most adverse drug reactions being dose-dependent. Administration of 20 mg/kg/day dosage resulted in somewhat greater reductions in seizure rates than the recommended maintenance dosage of 10 mg/kg/day, but with an increase in adverse reactions (pg. 2, label for Epidiolex®).

| | EPIDIOLEX | | |
|---|---|---|---|
| | 10 mg/kg/day | 20 mg/kg/day | Placebo |
| | N = 75 | N = 238 | N = 227 |
| Adverse Reactions | % | % | % |
| Hepatic Disorders | | | |
| Transaminases elevated | 8 | 16 | 3 |
| Gastrointestinal Disorders | | | |
| Decreased appetite | 16 | 22 | 5 |
| Diarrhea | 9 | 20 | 9 |
| Weight decreased | 3 | 5 | 1 |
| Gastroenteritis | 0 | 4 | 1 |
| Abdominal pain, discomfort | 3 | 3 | 1 |
| Nervous System Disorders | | | |
| Somnolence | 23 | 25 | 8 |
| Sedation | 3 | 6 | 1 |
| Lethargy | 4 | 8 | 2 |
| Fatigue, malaise, asthenia | 11 | 12 | 4 |
| Insomnia, sleep disorder, | 11 | 5 | 4 |
| poor quality sleep | | | |
| Irritability, agitation | 9 | 5 | 2 |
| Aggression, anger | 3 | 5 | <1 |
| Drooling, salivary | 1 | 4 | <1 |
| hypersecretion | | | |
| Gait disturbance | 3 | 2 | <1 |
| Infections | | | |
| Infection, all | 41 | 40 | 31 |
| Infection, viral | 7 | 11 | 6 |
| Pneumonia | 8 | 5 | 1 |
| Infection, fungal | 1 | 3 | 0 |
| Infection, other | 25 | 21 | 24 |
| Other | | | |
| Rash | 7 | 13 | 3 |
| Hypoxia, respiratory failure | 3 | 3 | 1 |

The dosage for oral CBD (Epidiolex®) is very high due to low bioavailability, the maintenance dose being 5 to 10 mg/kg twice daily (Epidiolex® label, pg. 1). Oral bioavailability of CBD is only 13 to 19% due to extensive first pass metabolism. Mechoulam et al., J Clin Pharmacol. 2002; 42:S11-S19. CBD is metabolized in the liver as well as in the intestines by CYP2C19 and CYP3A4 enzymes, and UGT1A7, UGT1A9, and UGT2B7 isoforms (Epidiolex® label, pg. 14). At steady state concentration, the major circulating moiety is 7-COOH-CBD, followed by parent drug CBD, 7-OH-CBD and 6-OH-CBD. The major metabolite 7-COOH-CBD is inactive but the minor metabolite 7-OH-CBD exhibits anticonvulsant effect similar to CBD.¹ The concentration of 7-COOH-CBD metabolite is 47-fold higher than CBD after 1500 mg CBD dose, and 97% of the total drug material measured. Taylor, CNS Drugs. 2018; 32:1053-1067. Therefore, the anticonvulsant activity is owed to only 3% of active molecules in systemic circulation, CBD and 7-OH-CBD, of which the majority is CBD (2%). Thus, there is a need for therapeutic methods for increasing the bioavailability of CBD. ¹ When tested in the Maximum Electroconvulsive Shock (MES) model in mouse.

Cobicistat, Tybost®, is indicated for the treatment of human immunodeficiency virus (HIV) infection in combination with other antiretroviral agents. Cobicistat is a component of two fixed-dose four-drug combination HIV treatments, Stribild® (elvitegravir/cobicistat/emtricitabine/tenofovir disoproxil) and Genvoya® (elvitegravir/cobicistat/emtricitabine/tenofovir alafenamide). Additionally, fixed-dose combination of cobicistat and protease inhibitors darunavir and azanavir are marketed as Prezcobix® and Evotaz®, respectively.

The pharmaco-enhancement of plasma concentrations of protease inhibitors by co-administration of boosters has long been an integral part of antiretroviral therapy for HIV. Nils von Hentig, HIV AIDS (Auckl). 2016; 8:1-16. Most HIV protease inhibitors are combined with low-dose ritonavir or cobicistat, which effectively inhibit the cytochrome-mediated metabolism of HIV protease inhibitors in the liver, thereby enhancing the plasma concentration and prolonging the dosing interval of the antiretroviral HIV protease inhibitors. Id. at 1. Cobicistat is similar to ritonavir to the extent that both are substrates for hepatic CYP3A metabolism. However, unlike ritonavir, cobicistat is reported to be a weak inhibitor of CYP2D6 (IC50=9.2 μmol/L) and does not inhibit CYP1A2, CYP2C8, CYP2C9, or CYP2C19 (IC50>25 μmol/L). Id. at 3; Mark Mascolini, “Cobicistat Has Little Impact on Key Drug-Metabolizing Enzyme,” 12th International Workshop on Clinical Pharmacology of HIV Therapy, Apr. 13-15, 2011, Miami.

When tested in the Maximum Electroconvulsive Shock (MES) model in mouse.

### SUMMARY

It has been found that effective inhibition of CBD metabolism requires inhibition of both CYP2C19 and CYP3A4 enzymes, as described herein. Further, CYP inhibition study described herein indicates that CBD has approximately three times higher affinity for CYP2C19 than CYP3A4. Without intending to be limited by any particular theory or mechanism of action, considering the average contribution of CYP2C19 in metabolizing CBD may be up to three times that of CYP3A based on their affinity, significant lowering of effective CBD dosage may be achieved by co-administration with compounds which are inhibitors of both CYP2C19 and CYP3A4 by virtue of higher bioavailability and higher effective plasma half-life of CBD upon such co-administration. Lowering the effective dosage of CBD would in turn reduce adverse reactions, especially hepatotoxicity, which is caused by dose-related elevations of liver transaminases (pg. 3-4, label for Epidiolex®).

In one aspect, the present invention provides a composition comprising:
cannabidiol, or pharmaceutically acceptable isomers, solvates and/or esters thereof; and
one or more compounds which are inhibitors of CYP2C19 and CYP3A4 enzymes.

In some embodiments, at least one compound which is an inhibitor of both CYP2C19 and CYP3A4 enzymes is co-administered with cannabidiol. In other embodiments, two or more compounds are co-administered with cannabidiol, wherein at least one compound is an inhibitor of CYP2C19 enzyme and at least another compound is an inhibitor of CYP3A4 enzyme.

In another aspect, the present invention provides a method for treating epilepsy, sleep deprivation, vomiting, nausea, psychosis, anxiety, depression, movement disorders, or other neuropsychiatric or neurogenic diseases in a patient comprising administering to the patient a therapeutically effective amount of:

cannabidiol, or pharmaceutically acceptable isomers, solvates and/or ester thereof; and

one or more compounds which are inhibitors of CYP2C19 and CYP3A4 enzymes.

In yet another aspect, the present invention provides a method for treating pain in a patient suffering from headache, migraines, rheumatoid arthritis, neuropathy, allodynia, overactive bladder, spasticity, multiple sclerosis, HIV, glioblastoma, cancer, or other acute or chronic pain conditions comprising administering to the patient a therapeutically effective amount of:

cannabidiol, or pharmaceutically acceptable isomers, solvates and/or ester thereof; and

one or more compounds which are inhibitors of CYP2C19 and CYP3A4 enzymes.

In another aspect, the invention provides a method for increasing plasma levels of CBD in a human being undergoing CBD treatment, comprising co-administering one or more compounds which are inhibitors CYP2C19 and CYP3A4 enzymes with a therapeutically effective amount of CBD, or pharmaceutically acceptable isomers, solvates and/or ester thereof.

In another aspect, the invention provides a method for decreasing hepatotoxicity in a human being undergoing CBD treatment, comprising co-administering a therapeutically effective amount of one or more compounds which are inhibitors of CYP2C19 and CYP3A4 enzymes, together with a therapeutically effective amount of CBD, or pharmaceutically acceptable isomers, solvates and/or ester thereof.

Further, cobicistat has now been unexpectedly found to inhibit not only CYP3A4, but also CYP2C19, as described herein. Thus, cobicistat can be used to alter pharmacokinetic profiles of therapeutic agents which are metabolized by CYP2C19 alone and therapeutic agents which are metabolized by both CYP2C19 and CYP3A4 enzymes.

In yet another aspect, the present invention provides a composition comprising:
cobicistat, or pharmaceutically acceptable isomers, salts, and/or solvates thereof; and
at least one therapeutic agent which is metabolized by CYP2C19 enzyme.

In another aspect, the present invention provides a method for inhibiting CYP2C19 in a patient in need thereof, comprising administering to the patient an amount of cobicistat, or pharmaceutically acceptable isomers, salts, and/or solvates thereof, effective for inhibiting CYP2C19 in the patient.

In another aspect, the present invention provides a method for increasing plasma levels of a therapeutic agent metabolized by CYP2C19 in a patient undergoing treatment with the therapeutic agent, comprising co-administering the therapeutic agent with a therapeutically effective amount of cobicistat, or pharmaceutically acceptable isomers, salts, and/or solvates thereof.

Both CYP3A4 and CYP2C19 are involved in primary metabolic pathways of several therapeutic agents. Blockage of both pathways maximizes metabolic inhibition because blockage of only one pathway can potentially divert the metabolism to the other pathway. Coadministration of cobicistat, which inhibits both CYP3A and CYP2C19, with such therapeutic agents is expected to significantly decrease the metabolism of the therapeutic agents, enabling significant lowering of dosage amount and frequency of administration of such therapeutic agents. Decreasing dosage amounts reduces adverse drug reactions, and decreasing dosing frequency increases patient compliance with the drug regimen.

In another aspect, the present invention provides a composition comprising:
cobicistat, or pharmaceutically acceptable isomers, salts, and/or solvates thereof; and
at least one therapeutic agent which is metabolized by CYP2C19 and CYP3A4.

In yet another aspect, the present invention provides a composition comprising:
cannabidiol, or pharmaceutically acceptable isomers, solvates and/or esters thereof; and
cobicistat, or pharmaceutically acceptable isomers, salts, and/or solvates thereof.

In another aspect, the invention provides a method for inhibiting CYP2C19 and

CYP3A4 in a patient in need thereof, comprising administering to the patient an amount of cobicistat, or pharmaceutically acceptable isomers, salts, and/or solvates thereof, effective for inhibiting CYP2C19 and CYP3A4 in the patient.

In yet another aspect, the present invention provides a method for increasing plasma levels of a therapeutic agent metabolized by CYP2C19 and CYP3A4 in a patient undergoing treatment with the therapeutic agent comprising co-administering the therapeutic agent with a therapeutically effective amount of cobicistat, or pharmaceutically acceptable isomers, salts, and/or solvates thereof.

### BRIEF DESCRIPTION OF THE DRAWINGS

FIG. 1 shows effect of cobicistat, 6,7-dihydroxybergamottin, lansoprazole and ketoconazole as CYP inhibitors on CBD metabolism in human liver microsomes.

FIG. 2 shows effect of fluoxetine, bergamottin, ticlopidine, omeprazole and omeprazole sulphone as CYP inhibitors on CBD metabolism in human liver microsomes.

### DETAILED DESCRIPTION

Compounds which are inhibitors of CYP2C19 and CYP3A4, and therapeutic agents metabolized by both CYP2C19 and CYP3A4 include CBD, cimetidine, chloramphenicol, voriconazole, fluvoxamine, isoniazid, 6,7-dihydroxybergamottin, lansoprazole, mixtures thereof, and pharmaceutically acceptable salts, isomers, solvates and/or ester thereof.

Compounds which are inhibitors of CYP2C19 and therapeutic agents metabolized by CYP2C19 include xenobiotics, including many proton pump inhibitors and antiepileptics. In particular, compounds which are inhibitors of CYP2C19 and therapeutic agents metabolized by CYP2C19 include clopidogrel, indomethacin, methadone, mephenytoin, phenylbutazone, ticlopidine, esomeprazole, lansoprazole, omeprazole, pantoprazole, rabeprazole, eslicarbazepine, felbamate, oxcarbazepine, efavirenz, diazepam, topiramate, fluoxetine, probenecid, modafinil, proguanil, and pharmaceutically acceptable salts, isomers, solvates and/or ester thereof.

The term “pharmaceutically acceptable salts” refers to salts prepared by treating the compound with pharmaceutically acceptable, non-toxic acids or bases. Suitable pharmaceutically acceptable acid addition salts of cobicistat include dihydrochloride, methanesulfonate, acetate, hydrobromide, salicylate, nitrate, dinitrate, pamoate, oxalate, p-toluene sulfonate, salicylate, tartarate, formate, and citrate salts.

Pharmaceutically acceptable isomers include all pharmaceutically active, non-toxic enantiomers, diastereomers, and geometric isomers. Pharmaceutically acceptable solvates include hydrates and solvates with pharmaceutically acceptable solvents, such as alcohols.

Cannabidiol, cobicistat, compounds which are inhibitors of CYP2C19 and/or CYP3A4, and therapeutic agents metabolized by CYP2C19 and/or CYP3A4, or isomers, salts, solvates and/or esters thereof, are commercially available or may be manufactured by methods well known in the art. For example, cobicistat may be prepared by methods disclosed in U.S. Pat. No. 9,975,864 B2 or Xu et al., ACS Med. Chem. Leh. 2010,1(5):209-213, S10-S14; and CBD may be prepared by methods disclosed in U.S. Pat. Nos. 2,304,669 A, 5,227,537 A, or 7,674,922 B2.

The amount of compounds which are inhibitors of both CYP2C19 and/or CYP3A4, and therapeutic agents metabolized by CYP2C19 and/or CYP3A4 to be used according to the compositions and methods of the invention depends on the desired therapeutic effect, and can thus vary within a wide range, but is generally significantly lower than the amount required to achieve the same effect via monotherapy or without using the claimed combination of active ingredients. A person skilled in the art can readily determine the appropriate amounts based on the dosages for monotherapy known in the art. Generally, therapeutically effective amount of a therapeutic agent or compound is an amount sufficient to elicit any of the beneficial effects of the therapeutic agent or compound in a patient. For example, therapeutically effective amount of cobicistat, or pharmaceutically acceptable isomers, salts, and/or solvates thereof, is an amount sufficient to inhibit or reduce the metabolism of therapeutic agents metabolized by CYP2C19 and/or CYP3A4.

Compounds which are inhibitors of CYP2C19 and CYP3A4 increase the plasma concentration of CBD by inhibiting the metabolism of CBD. Cobicistat increases the plasma concentration of compounds which are inhibitors of CYP2C19 and/or CYP3A4 by inhibiting their metabolism.

CBD shows dose dependent absorption. Pharmacokinetic data², of a range of single doses from 200 mg to 6000 mg shows that the absorption of CBD decreases significantly with increasing dosage, likely due to the limited solubility of CBD in gastrointestinal fluid. CBD is highly hydrophobic-Log P (partition coefficient) is 6.1 and water solubility is 0.0126 mg/m1³. ² Taylor 2018, CNS Drugs 2018;32:1053-1067; Clinical pharmacology and biopharmaceutics review(s), Epidiolex®³ Drug bank:Cannabidiol_https://go.drugbank.com/drugs/DB09061

| Parameter | 200 mg | 750 mg | 1500 mg | 3000 mg | 4500 mg | 6000 mg |
|---|---|---|---|---|---|---|
| CBD AUC | 499 | 1070 | 1444 | 2669 | 3215 | 3696 |
| (ng · h/mL) | | | | | | |
| % Relative | 100 | 57 | 39 | 36 | 29 | 25 |
| Bioavailability | | | | | | |

Overall, CBD bioavailability depends on two factors-first pass metabolism (mainly by CYP2C19 and CYP3A4) and dose amount. CYP3A4 and CYP2C19 inhibitors are expected to increase bioavailability by more than 3 fold by inhibiting first pass metabolism. Further, the resulting reduction in dose would also increase absorption across the gastrointestinal tract multifold.

Significant interaction of CBD with food has been reported⁴ due to limited solubility and consequent low absorption of CBD at higher doses. Hence, by reducing the dose of CBD by co-administering CYP3A4 and CYP2C19 inhibitors, food interaction of CBD can be significantly reduced making the pharmacokinetic profile more predictable, which in turn improves the safety profile of CBD. ⁴ Taylor 2018, CNS Drugs 2018;32:1053-1067; Clinical pharmacology and biopharmaceutics review(s), Epidiolex®

Further, by inhibiting the metabolism of CBD, compounds which are inhibitors of CYP2C19 and CYP3A4 may increase the effective half-life of CBD, thereby decreasing the frequency of dosing. Cobicistat may increase the effective half-life of therapeutic agents metabolized by CYP2C19 and/or CYP3A4 by inhibiting their metabolism, thereby decreasing the frequency of their dosing.

For example, the short half-life of cobicistat of 3 to 5 hours makes it suitable for co-administration with CBD, which has a half-life of 10 to 17 hours, for once daily dosage treatment by extending the effective half-life of CBD (t½,eff) to 15-22 hours through inhibition of metabolism of systemically available CBD. Once daily treatment increases patient compliance. Moreover, when patients are on another concurrent medication metabolized by or inhibited by either or both CYP3A4 and CYP2C19 enzymes, the administration of CBD and the concurrent medication can be suitably staggered throughout the day to minimize drug-drug interactions by administering CBD once daily with cobicistat.

The total daily dosage of CBD administered with cobicistat or other compounds which are inhibitors of CYP2C19 and/or CYP3A4 may be, for example, half, a third, a fourth, a fifth, a tenth, a fifteenth, or a twentieth of the regular dosage amount of CBD when administered alone. Amount of CBD used in the treatment methods of the invention may be, for example, in the range of 1 mg/kg once a day to 20 mg/kg once a day, or from 0.5 mg/kg twice daily to 10 mg/kg twice daily. In some embodiments, the composition according to the invention comprises 25 mg to 4000 mg, 50 mg to 2000 mg, or 100 mg to 1000 mg of CBD.

The amount of cobicistat used in the treatment methods of the invention may be, for example, in the range of 100 mg once a day to 1000 mg once a day, or 50 mg twice a day to 500 mg twice a day. In some embodiments, the composition according to the invention comprise cobicistat in an amount of between 100 mg and 1000 mg.

The coadministration or administration of CBD or cobicistat according to the invention may be performed simultaneously with, before, or after the administration of the compounds which are inhibitors of CYP2C19 and CYP3A4, or the therapeutic agents metabolized by CYP2C19 and/or CYP3A4, respectively. According to one alternative the compounds which are inhibitors of CYP2C19 and CYP3A4 or the therapeutic agent metabolized by CYP2C19 and/or CYP3A4 is administered first and CBD or cobicistat is administered second. According to another alternative, CBD or cobicistat is administered first and the compounds which are inhibitors of CYP2C19 and CYP3A4 or the therapeutic agent metabolized by CYP2C19 and/or CYP3A4 is administered second. According to some embodiments, the administration of CBD or cobicistat may be performed up to two hours before or after the administration of the compounds which are inhibitors of CYP2C19 and CYP3A4 or the therapeutic agents metabolized by CYP2C19 and/or CYP3A4. For example, CBD or cobicistat may be administered 15 minutes, 30 minutes, 45 minutes, one hour, or one and a half hour before or after the administration of the compounds which are inhibitors of CYP2C19 and CYP3A4 or the therapeutic agents metabolized by CYP2C19 and/or CYP3A4 to maximize or control the metabolism of the other drug/therapeutic agent.

Accordingly, the claimed combination of active ingredients, CBD, cobicistat, the compounds which are inhibitors of CYP2C19 and CYP3A4, the therapeutic agent metabolized by CYP2C19 and/or CYP3A4, may be included in a single pharmaceutical composition or contained separately in two independent pharmaceutical compositions.

The active ingredients may be formulated together with pharmaceutically acceptable carriers and/or excipients as a tablet, or capsule. The compositions are prepared according to the usual methods known in the art.

When the claimed combination of active ingredients are contained separately in two independent pharmaceutical compositions, the two pharmaceutical compositions may constitute different dosage forms and routes of administration. For example, one of the pharmaceutical compositions may be a tablet and the other may be capsule. In particular, cobicistat may be formulated together with pharmaceutically acceptable carriers and/or excipients as a coated or uncoated tablet, or a capsule. CBD, the therapeutic agent metabolized by CYP2C19 and/or CYP3A4, or the compounds which are inhibitors of CYP2C19 and CYP3A4 may be formulated together with pharmaceutically acceptable carriers and/or excipients as an oral solution or suspension, tincture, self-microemulsifying drug delivery system, chewable gummy, gel capsule, sublingual drops, tablets, vaping oil, smoking compositions, or any other means of oral, topical or nasal administration.

The composition comprising CBD may be administered orally, sublingually, topically or via inhalation. The composition comprising therapeutic agents metabolized by CYP2C19 and/or CYP3A4 may be administered orally, sublingually, topically, via inhalation, or any other route of administration. The composition comprising compounds which are inhibitors of CYP2C19 and/or CYP3A4 may be administered orally, sublingually, topically, via inhalation, or any other route of administration. The composition comprising cobicistat alone or in combination with compounds which are inhibitors of CYP2C19 and/or CYP3A4 may generally be administered orally.

### EXPERIMENTAL

### Cytochrome P450 Inhibition in Human Liver Microsomes (HLM)

### Example 1

The direct inhibition potential of a number of CYP3A4 and CYP2C19 inhibitors on the metabolism of CBD at a predetermined Km⁵ (the substrate concentration at which reaction rate is 50% of Vmax, where Vmax is maximum rate achieved by the system) using pooled human liver microsomes. ⁵ Km is a measure of the affinity an enzyme has for its substrate, as the lower the value of Km, the more efficient the enzyme is at carrying out its function at a lower substrate concentration.

### Km/Vmax

HLM diluted in potassium phosphate buffer were mixed with compound working solution and 5× cofactor (0.44 mM NADP, 5.5 mM G6P, 0.4 U/mL G6PDH) to achieve final concentrations of 0.033, 0.1, 0.33, 1, 3.3, 10, 33, and 100 μM test compound, 1 μM for positive control midazolam, and 0.1 mg/mL for liver microsomes. Spiked liver microsomes were incubated at 37° C. with 5% CO₂ at 200 rpm. At 0, 15, 45, 90, and 120 minutes, 50 μL aliquots of spiked liver microsomes were collected and quenched with 200 μL cold stop solution (acetonitrile containing internal standard) in a deep 96-well collection plate. At the end of the incubation, the collection plate was vortexed at 1700 rpm for 3 minutes and centrifuged at 3500 rpm for 10 minutes. The resulting supernatant was mixed with water (1:1, v/v) in a new deep 96-well plate. The samples were then analyzed using liquid chromatography tandem mass spectrometry (LC-MS/MS). The Km and Vmax values were determined from regression analysis of test compound disappearance and metabolite formation versus test compound concentration.

### IC50 Determination

Microsome mixtures were prepared in 100 mM KPi with 100X positive control working solutions and 5X cofactors; and reactions were initiated with 5X substrate working solutions for a final volume of 250 μL. The final incubation concentrations were 0.1 mg/mL microsomal protein; 1X cofactor; 0.033, 0.1, 0.33, 1, 3.3, 10, 33 μM test inhibitor; 0.5% DMSO, with inhibitor (positive control) and substrate concentrations as listed in the tables below. Samples were incubated at 37° C. and 5% CO2 with orbital shaking at 200 rpm for 15 minutes. Reactions were stopped by adding 500 μL of cold stop solution (acetonitrile containing internal standard). The samples were vortexed at 1700 rpm for 3 minutes and centrifuged at 3500 rpm for 15 minutes. The resulting supernatant was mixed with water (3:1, v/v) in a new deep 96-well plate. The samples were analyzed by LC-MS/MS for substrate disappearance and metabolite formation.

| | | | | Final |
|---|---|---|---|---|
| | | | Working | Conc. in |
| | | | Conc. | reaction |
| Isoform | Substrate | Stock Conc.(mM) | (μM) | (μM) |
| NA | Cannabidiol | 10 | 8.2 | 1.64 |
| 2C19 | S-mephenytoin | 50 | 125 | 25 |
| 3A4 | Midazolam | 7.5 | 6.25 | 1.25 |

| | |
|---|---|
| Ticlopidine (2C19) | Ketoconazole (3A4) |

| Working | Final Conc. in | Working | Final Conc. in |
|---|---|---|---|
| Conc.(mM) | reaction (μM) | Conc.(mM) | reaction (μM) |
| 0 | 0 | 0 | 0 |
| 0.0033 | 0.033 | 0.000033 | 0.00033 |
| 0.01 | 0.1 | 0.0001 | 0.001 |
| 0.033 | 0.33 | 0.00033 | 0.0033 |
| 0.10 | 1 | 0.001 | 0.01 |
| 0.33 | 3.3 | 0.0033 | 0.033 |
| 1.0 | 10 | 0.01 | 0.1 |
| 3.3 | 33 | 0.033 | 0.33 |

The results are provided in the table below:

| | HLM CYP inhibition IC50 (μM), (n = 1) |
|---|---|
| Test Inhibitor | CBD |
| Cobicistat | 17.5125 |
| Bergamottin | >33 |
| 6,7-dihydroxybergamottin | 23.3260 |
| Fluoxetine | >33 |
| Omeprazole | >33 |
| Omeprazole Sulphone | >33 |
| Lansoprazole | 19.9832 |
| Ketoconazole - CBD | 26.9435 |
| Ticlopidine - CBD | >33 |

| | | Average |
|---|---|---|
| Positive Control | IC50 | Historical IC50 |
| Ketoconazole | 0.0314 | 0.0229 |
| Ticlopidine | 0.690 | 0.591 |

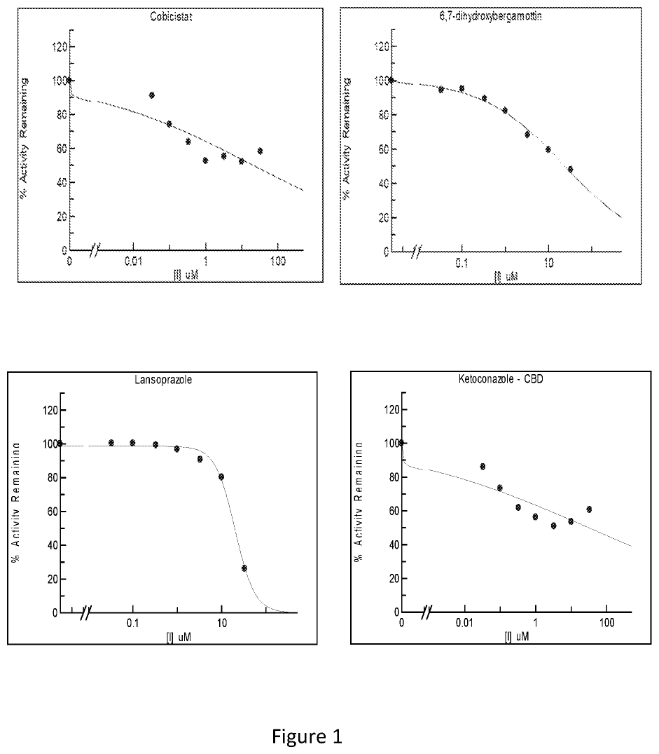

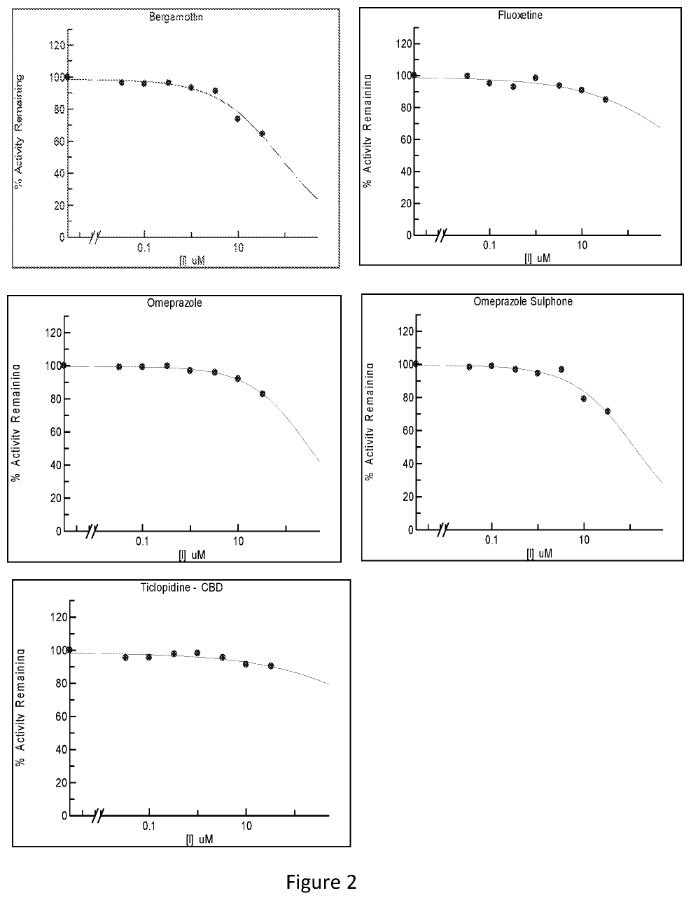

The data shows that cobicistat is a strong inhibitor of CBD metabolism. 6,7-dihydroxybergamottin and lansoprazole were also found to be effective inhibitors of CBD metabolism. 6,7-dihydroxybergamottin is known for inhibiting not only CYP3A4 but also CYP2C19. H. Seki et al. Drug Metab. Pharmacokinet. 2019;34(3)181-186. Lansoprazole is known to be primarily an inhibitor of CYP2C19 and CYP3A4. Meyer et al. Eur J Gastroenterol Hepatol 1996; 8 (Suppl. 1): S21 5. The results also demonstrate that ticlopidine and fluoxetine, inhibitors of CYP2C19 but not CYP3A4, are unable to inhibit metabolism of CBD. The results also show that ketoconazole, an inhibitor of CYP3A4, also inhibits CBD metabolism but is less potent than cobicistat, 6,7-dihydroxybergamottin and lansoprazole. Cobicistat was found to be 1.53 times more potent inhibitor of CBD metabolism than ketoconazole, a sole inhibitor of CYP3A4. Graphs in FIGS. 1 and 2 show the effect of the tested CYP inhibitors on CBD metabolism in human liver microsomes.

It can be inferred that despite CYP2C19 based metabolism being the major pathway, the metabolism of CBD diverts to other pathways, such as CYP3A4 based metabolism when CYP2C19 is inhibited. The results demonstrate that CYP2C19 inhibition alone does not inhibit metabolism of CBD and inhibiting CYP3A4 alone does not appear to be sufficient. Thus, the combined inhibition of both CYP3A4 and CYP2C19 pathways is necessary for effective inhibition of CBD metabolism.

A previous clinical kinetic study has reported that Ketoconazole, a sole inhibitor of CYP3A4, increases AUC(0-t) of CBD by 2.65 times. Stott et al. SpringerPlus 2013, 2:236. Hence, cobicistat is expected to increase AUC of CBD by significantly more than three-fold.

### Example 2

A cytochrome P450 (CYP) direct inhibition study was conducted to evaluate the direct inhibition potential of test compounds on human hepatic (CYP) isoforms using pooled human liver microsomes and isoform-specific probe substrates. Incubation mixtures were prepared with the following:

(a) potassium phosphate buffer;

(b) 0.1 mg/mL microsomal protein;

(c) isoform-specific probe substrates;

(d) specific concentrations of the test compounds, blank solvent, or positive controls (isoform-specific inhibitors); and

(e) an NADPH-regenerating cofactor solution.

The mixtures were incubated at 37° C. and 5% carbon dioxide with orbital shaking at 200 rpm for 5-20 minutes depending on the substrate. Upon completion of the incubation, the reactions were quenched with an acetonitrile solution containing an internal standard. The samples were processed and analyzed by LC-MS/MS to monitor for substrate metabolite formation. The enzyme activity in the presence of the compounds were normalized with the enzyme activity in the absence of the compounds and expressed as a percentage of activity. The inhibitory potential (IC50) of the compounds were determined using nonlinear regression of percentage of activity versus compound concentration. The test results are provided in the table below:

| | HLM CYP inhibition IC50 (μM), (n = 1) | | | |
|---|---|---|---|---|
| Test | CYP2C19 | CYP2D6 | CYP3A4 | CYP3A4 |
| Compound | (S-Mephenytoin*) | (Dextromethorphan*) | (Midazolam*) | (Testosterone*) |
| Cannabidiol (CBD) | 0.8738 | 8.0542 | 2.6155 | 2.7895 |
| Cobicistat | 0.3314 | 0.4792 | 0.0333 | 0.0519 |
| Fluvoxamine | 12.2707 | 5.7870 | >33 | 29.1959 |
| Fluoxetine | 0.2541 | 6.8370 | >33 | 21.7532 |
| Positive Control | 0.6658 | 0.0294 | 0.0143 | 0.0177 |
| | (Ticlopidine#) | (Quinidine#) | (Ketoconazole#) | (Ketoconazole#) |
| Average Historical | 0.4819 | 0.0411 | 0.0181 | 0.0157 |
| Control | | | | |

*Compound used as a substrate for enzyme.
#Compound used as positive control for enzyme inhibition.

The data shows that CBD has approximately three times higher affinity for CYP2C19 than CYP3A4.

1. A composition comprising:
cannabidiol, or pharmaceutically acceptable isomers, solvates and/or esters thereof; and
one or more compounds which are inhibitors of CYP2C19 and CYP3A4 enzymes.

2. The composition of claim 1 wherein the composition comprises one or more compounds which are inhibitors of both CYP2C19 and CYP3A4 enzymes.

3. The composition of claim 1 wherein the composition comprises at least one compound which is inhibitor of CYP2C19 enzyme and at least another compound which is inhibitor of CYP3A4 enzyme.

4. The composition of claim 2 wherein the compound is selected from the group consisting of cobicistat, 6,7-dihydroxybergamottin, lansoprazole, cimetidine, chloramphenicol, voriconazole, fluvoxamine, isoniazid, mixtures thereof, and pharmaceutically acceptable salts, isomers, solvates and/or ester thereof.

5. The composition of claim 1 further comprising one or more pharmaceutically acceptable carriers or excipients.

6. A method for treating epilepsy, sleep deprivation, vomiting, nausea, psychosis, anxiety, depression, movement disorders, and other neuropsychiatric or neurogenic diseases in a patient comprising administering to the patient a therapeutically effective amount of the composition of claim 1.

7. A method for treating pain in a patient suffering from headache, migraines, rheumatoid arthritis, neuropathy, allodynia, overactive bladder, spasticity, multiple sclerosis, HIV, glioblastoma, cancer, and other acute or chronic pain conditions comprising administering to the patient a therapeutically effective amount of the composition of claim 1.

8. A method for decreasing hepatotoxicity in a human being undergoing cannabidiol treatment, comprising administering to the human being a therapeutically effective amount of the composition of claim 1.

9. A method for increasing cannabidiol plasma levels in a patient undergoing cannabidiol treatment comprising administering to the human being a therapeutically effective amount of the composition of claim 1.

10. (canceled).

11. (canceled).

12. A method for increasing cannabidiol plasma levels in a patient undergoing cannabidiol treatment comprising administering
a therapeutically effective amount of the composition of claim 4.

13. A composition comprising:
cobicistat, or pharmaceutically acceptable isomers, salts, and/or solvates thereof;
and
at least one therapeutic agent which is metabolized by CYP2C19.

14. The composition of claim 13 wherein the therapeutic agent metabolized by CYP2C19 is also metabolized by CYP3A4.

15. The composition of claim 13 wherein the therapeutic agent is selected from the group consisting of clopidogrel, indomethacin, methadone, mephenytoin, phenylbutazone, ticlopidine, esomeprazole, lansoprazole, omeprazole, pantoprazole, rabeprazole, eslicarbazepine, felbamate, oxcarbazepine, efavirenz, diazepam, topiramate, fluoxetine, probenecid, modafinil, proguanil, and pharmaceutically acceptable isomers, salts, solvates and/or ester thereof.

16. The composition of claim 14 wherein the therapeutic agent is selected from the group consisting of cannabidiol, cimetidine, chloramphenicol, voriconazole, fluvoxamine, isoniazid, cidoxepin, 6,7-dihydroxybergamottin, lansoprazole, mixtures thereof, and pharmaceutically acceptable isomers, salts, solvates and/or ester thereof.

17. The composition of claim 14 wherein the therapeutic agent is selected from the group consisting of:
cannabidiol, or pharmaceutically acceptable isomers, solvates and/or esters thereof.

18. The composition of claim 13 further comprising one or more pharmaceutically acceptable carriers or excipients.

19. A method for inhibiting CYP2C19 in a patient in need thereof, comprising administering to the patient an amount of cobicistat, or pharmaceutically acceptable isomers, salts, and/or solvates thereof, effective for inhibiting CYP2C19 in the patient.

20. The method of claim 19 wherein the method involves concomitantly inhibiting both CYP2C19 and CYP3A4 in a patient in need thereof, and the method comprises administering to the patient an amount of cobicistat, or pharmaceutically acceptable isomers, salts, and/or solvates thereof, effective for inhibiting both CYP2C19 and CYP3A4 in the patient.

21. A method for increasing plasma levels of a therapeutic agent metabolized by CYP2C19 in a patient undergoing treatment with the therapeutic agent comprising administering to the patient a therapeutically effective amount of the composition of claim 13.

22. A method for increasing plasma levels of a therapeutic agent metabolized by CYP2C19 and CYP3A4 in a patient undergoing treatment with the therapeutic agent comprising administering to the patient a therapeutically effective amount of the composition of claim 14.
